(19) United States
(12) Patent Application Publication
(10) Pub. No.: US 2013/0023811 A1
(43) Pub. Date: Jan. 24, 2013

(54) BANDAGE FOR THE PREVENTION OF A BOIL

(71) Applicant: G. William Haas, New York, NY (US)

(72) Inventor: Herbert B. L. Silverman, New York, NY (US)

(21) Appl. No.: 13/547,222

(22) Filed: Jul. 12, 2012

Related U.S. Application Data
(60) Provisional application No. 61/572,653, filed on Jul. 20, 2011.

(51) Int. Cl. A61F 13/02; A61F 13/00

(52) U.S. Cl. 602/43

(57) ABSTRACT

A bandage for aborting an infection caused by staph bacterium (a “staph spot”) at its incipient stage preventing the emergence of a boil, comprising: a pad having a central aperture; an adhesive covering strip long enough to cover the pad attached to the top thereof at only one point for attachment to the subject's skin having a width less than that of the central aperture to form an air chamber and at least one tunnel which passes through the wall of the pad from outside to inside to the central aperture. The bandage is used by affixing the pad and one end of the adhesive covering strip to the skin, with the central aperture encircling the staph spot without touching it and taping down the other end of the strip for affixing the strip at both its ends from atop the pad onto the subject's skin.

### FIELD OF THE INVENTION

This application claims priority of U.S. provisional application Ser. No. 61/572,653, filed Jul. 20, 2011, the entirety of which is incorporated herein by reference thereto.

The present invention relates to a medical device—a bandage—which is to be used in hospitals, physician's offices and homes as a means of aborting an infection of the staphylococcus aureus (“staph”) bacterium, thereby preventing emergence of a boil.

### BACKGROUND OF THE INVENTION

Boils and carbuncles are skin infections usually caused by staph bacteria digging into the skin around the root of a hair (or, in the case of carbuncles, hairs. A carbuncle is actually a cluster or group of boils in the skin and in the tissue beneath, usually caused by staphylococcus aureus, with the formation of drainage ducts and necrotic tissue). In appearance, a boil is a red, swollen, severely painful lump under the surface of the skin and can grow to the size of a golf ball. Such an infection will form a pocket of pus, a viscous, yellowish fluid consisting of bacteria, dead skin cells and dead infection-fighting white blood cells. As the infection progresses, a whitish tip, known as a point or a head, will appear atop the boil at its center. It is through this head, when opened by medical intervention or otherwise, that the boil's pus will drain.

Traditionally, small boils are treated by opening of the head by means of moist heat, usually applied via a warm, wet washcloth for twenty to thirty minutes at a time, three or four times a day. Such measures will cause the head to open so that the pus can be squeezed out. Once the boil has been so drained, it should be covered with a clean bandage, which will protect the opened skin and collect any further drainage of pus. The affected area should be washed several times daily with anti-bacterial soap in order that spreading of the infection may be prevented. Anyone who has given the patient attention should also wash his or her hands with antibacterial soap.

Boils commonly occur on the buttocks, face and neck and in the armpits and groin. Carbuncles are deeper and are found on the back of the neck and on the shoulders, hips and thighs. Carbuncles are chiefly common in diabetics and in middle-aged and elderly men.

Large boils and carbuncles are normally treated with antibiotics. Generally, the physician will first drain the infected area of pus through a small incision. Such treatment will relieve pain, speed recovery and limit scar formation. If the infection is deep, the physician may fill the pocket from which the pus has been drained with sterile gauze as a means of keeping the incision open, while allowing any remaining pus to drain. The patient will likely need to visit the physician several times to have the dressing refreshed. The pocket will heal slowly, becoming more shallow over time until it is no more than a superficial wound.

Obviously, total avoidance of boils is infinitely to be preferred to suffering boils and having to deal with them. The device of the instant invention, when applied promptly upon the appearance of the characteristic itchy or stinging small spot manifesting the actuality of a staph infection, has been found unfailingly by the inventor to abort the staph infection and thereby to prevent development of a boil.

In instances in which an actual boil has had to be treated, the conventional treatment encounters significant difficulties: the bandage and dressing, if applied tightly, will deprive the affected area of oxygen and at the same time slow the drainage of pus. If the bandage and dressing are more loosely applied, allowing some freedom of motion, they will rub against the affected area during the day's activities, causing irritation. The instant invention, having, as it does, the structure of a small air chamber, keeps the wound constantly aerated while at the same time shielding it from any external irritants.

Other objects, features and advantages of the invention will be apparent from the detailed description thereof and the accompanying drawings, which should be considered in an illustrative and non-limiting sense, as follows.

### BRIEF DESCRIPTION OF THE DRAWINGS

FIG. 1 is a top view of the pad of the invention in a preferred embodiment.

FIG. 2 is a cross-sectional view of the pad along the 2-2 line.

FIG. 3 is a side view of the device with the pad and one end of the adhesive strip, fully affixed to the skin of the patient.

FIG. 4 is identical to FIG. 3 except that part of the strip covering the pad and the loose end of the strip, shown in FIG. 3, is, in FIG. 4, no longer loose but is taped down firmly with the loose end of the strip now affixed to the skin of the patient.

FIG. 5 is a top view of the device as fully affixed to the skin of the patient.

### DETAILED DESCRIPTION OF THE INVENTION

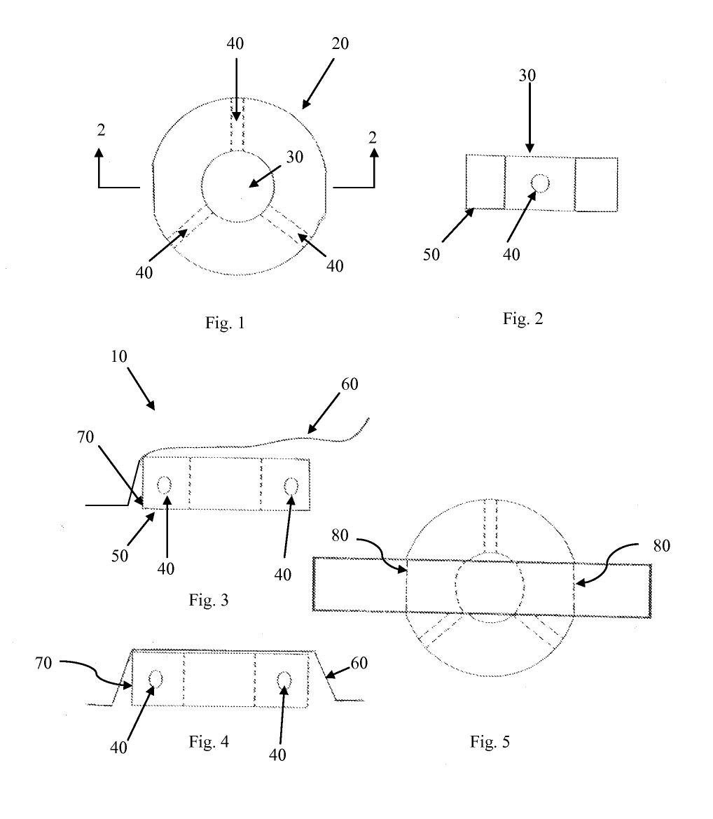

The device 10 consists of a) a thick pad (all FIGS., item 20 in FIG. 1) (of foam, felt, rubber or some other suitable substance) preferably circular in shape except that—the more neatly to accommodate taping down of the adhesive strip 60 (see “b)”, immediately below) to the top of the pad—two small sections, providing straightedges 80, are cut from the circle in opposite positions, each cut being made along a secant having a length equal to the width of the strip, and said pad having a central through aperture 30, circular in shape and concentric with the pad and passing entirely through the pad from top to bottom; and the device consists further of b) an adhesive strip, 60 (FIG. 3), which is attached, by stitching or adhesion, to the top of the pad at only one point, 70 (FIGS. 3 and 4). The adhesive strip, the other constituent of this device, is attached to the top of the pad near one of its ends only, the other end being left loose so that the patient may raise it and see through the aperture of the pad in order to be enabled to position the pad on the skin—only then does he affix the loose end to his skin. Thus, the purpose of one-pointed attachment is that of affording the patient the necessary convenience of being able to lift the other end of the strip in order to be enabled to see through the central aperture 30 as he positions the device on his skin.

The patient, in affixing the pad and one end of the strip of this device adhesively to his skin, positions them so that the central aperture of the pad encircles the staph spot without the pad's touching said spot. He then tapes down onto his skin the other end of the adhesive strip 60, thereby not only accomplishing additional security of adhesion but also effecting a small air chamber, of which the pad and the strip constitute, respectively, the wall and the roof. Such a chamber keeps the staph spot constantly aerated by virtue of the ventilated condition of the chamber while, at the same time, the infection is shielded, essentially by the strip, from external irritation, primarily the friction of the patient's clothing.

Ventilation of the air chamber is accomplished, in part, in consequence of the fact that the width of the covering strip is somewhat less than the diameter of the central aperture, thus leaving the aperture partly exposed to circulation of atmospheric air. Ventilation is further enhanced by the provision of at least one, but preferably not fewer than three, tunnels spaced evenly apart, and passing entirely through the wall of the pad from outside to inside, reaching the central aperture and thereby allowing access of outside air into the aperture. For the sake of rigidity of the tunnels, preventing their collapse when, for example, the patient sits upon the bandage, the tunnels are lined with firm tubing of plastic or some other suitable substance.

With regard to adhesion: the bottom surface, 50—FIGS. 2 and 3—of the pad is coated with an adhesive which, like the adhesive of the strip, is protected until use by peelable paper—so called “release paper.” As an alternative means of affixture for the pad, a system of lacing may be used. In situations in which the infection has appeared on an exposed area of the body—say the forearm—rather than under the clothing, a combination of lacing and adhesion may be desirable for purposes of added security.

If this device has been applied promptly as soon as the staph infection has manifested itself in the form of the characteristic spot on the skin, the infection will abort after a period of several days and no boil will emerge.

As stated above, in instances in which a boil has had to be opened and treated, the instant invention, having, as it does, the structure of a small roofed air chamber, will be very effective in keeping the wound constantly aerated while at the same time protecting it from external irritants such as friction of the patient's clothing.

As to substance: the pad can be made of foam, felt or rubber or any suitable material which will not, in some way, have an effect through the adhesive and irritate the skin or lead to some other undesirable consequence.

The invention is directed to a bandage for aborting an infection of the staph bacterium (a “staph spot”) at its incipient stage, thereby preventing the emergence of a boil or a carbuncle. The device comprises a) a pad having a through central aperture and preferably an adhesive on the bottom surface thereof for affixing the device to the skin of an affected subject; and b) an adhesive strip, comparable to the familiar adhesive-strip bandages currently in use in cases of minor cuts or abrasions, attached to the top of the pad at only one point until the device is used. The apertured pad and the covering strip together form a little air chamber.

The invention is also directed to a method of accomplishing the abortion and prevention of the infection by a) first affixing the pad and one end of the strip to the skin, so positioned that the through central aperture, at its bottom, encircles the staph spot without touching it; and then b) taping down the other end of the strip, after the pad has been positioned. Thus the strip is now taped down at both its ends from atop the pad onto the subject's skin. The air-chamber structure affords aeration of the staph spot while shielding the spot from external irritants.

The invention provides two means of ventilating the chamber formed by the apertured pad (as the chamber's wall) and the strip (as the chamber's roof): the first of these means is the partly open roof of the chamber, admitting circulating air, such opening being achieved by having the width of the adhesive strip slightly less than the diameter of the central aperture. The second means of ventilation is that afforded by the provision, penetrating through the side of the pad from outside to inside and reaching the central aperture, of at least one but preferably three or more tunnels spaced evenly apart, through which atmospheric air circulates into the chamber.

The device of the invention is also useful in instances in which a developed boil has had to be opened and drained and requires, during convalescence, aeration as well as shielding from irritants. The device is well qualified, by virtue of its structure as a small and roofed air chamber, to provide both these conditions.

The device of the invention can also be used to shield small, painful cuts, abrasions, insect bites, etc. to provide shielding and protection from irritants such as clothing.

The device of the invention may be made in various sizes in order to accommodate the underlying condition, boil, carbuncle, insect bite, cut or abrasion.

The pad 10 can be circular, square, oval or otherwise and from about 1 to 5 inches in length or diameter. The opening 30 for providing ventilation will occupy about ⅓ to ½ the area thereof and will be centrally located. The adhesive strip employing the conventional adhesive will be long enough to cover over the pad and to provide for attachment to the patient's skin as described, but will be of a width less than that of the central opening. This exposes the aperture to circulation of atmospheric area which is enhanced by at least one tunnel and preferably at least two spaced evenly apart and passing entirely through the wall of the pad from outside to inside, reaching the central opening.

The devices of the invention are provided preferably individually-wrapped and sealed until use.

I claim:

1. A device for preventing emergence of a boil or a carbuncle from a “staph spot” on the skin of an affected subject, said device comprising a pad having a central through aperture; and said device comprising also a strip means for additional security of affixture of said pad to said skin, said strip attached at only one point of the top surface of said pad, and with the lower end of said central through aperture encircling said staph spot, without the pad being in contact with said staph spot.

2. A device according to claim 1, wherein said pad further contains at least one tunnel through its wall connecting said central aperture to the atmosphere, and wherein at least one of said tunnels is not sealed off by said affixing strip means.

3. A device according to claim 2, wherein said affixing means is an adhesive strip applied over said pad wherein said central aperture is substantially but not fully closed and wherein at least one of said tunnels is at least partially left open to the atmosphere.

4. A device according to claim 3, wherein said affixing means further comprises at least a member selected from the group consisting of laces attached to said pad and an adhesive installed on the under side of said pad, the side in contact with said skin during use.

5. A method for treating a staph spot threatening emergence of a boil or a carbuncle using a device according to claim 1, comprising the steps of:
a) applying said pad over said skin with said lower end of said central aperture encircling said staph spot and wherein said pad is not in contact with said staph spot;
b) affixing said pad securely to said skin by means of the pad's adhesive and that of the adhesive strip located on the top of the pad; and
c) removing said pad after a period of time.
